(19) United States
(12) Patent Application Publication
(10) Pub. No.: US 2026/0260708 A1
(43) Pub. Date: Sep. 3, 2026

(54) PREDICTING PROPERTIES OF SINGLE VARIABLE DOMAINS USING MACHINE-LEARNING MODELS

(71) Applicant: Sanofi, Paris (FR)

(72) Inventors: Abhinav GUPTA, Paris (FR); Yu QIU, Paris (FR); Sven JAGER, Paris (FR); Alejandro CORROCHANO NAVARRO, Paris (FR); Michael BAILEY, Paris (FR); Jose Antonio AMMENGUAL RIGO, Paris (FR); Michael HEINZINGER, Paris (FR); Norbert FURTMANN, Paris (FR); Jose FLORES CANALES, Paris (FR)

(21) Appl. No.: 19/489,487

(22) PCT Filed: Jun. 5, 2024
(86) PCT No.: PCT/EP2024/065413

§ 371 Date: Dec. 2, 2025

(30) Foreign Application Priority Data
Jun. 5, 2023 (EP) 23305893.2

(51) Int. Cl. G16B 40/20; G16B 30/10

(52) U.S. Cl. CPC G16B 40/20; G16B 30/10

(57) ABSTRACT

Methods, computer systems, and apparatus, including computer programs encoded on computer storage media, for predicting ISVD molecule properties. The system obtains a token sequence representing the ISVD molecule, generates an input vector by numerically encoding the amino acid sequence, generates an embedded feature vector by processing the input vector using an embedding machine-learning model having a first set of model parameters, and processes the embedded feature vector using a property-prediction machine-learning model to generate an output that predicts one or more properties of the ISVD molecule.

### CROSS-REFERENCE TO RELATED APPLICATION

This application is the national stage entry of International Patent Application No. PCT/EP2024/065413, filed on Jun. 5, 2024, and claims priority to European Patent Application No. EP23305893, filed on Jun. 5, 2023, the disclosure of which is hereby incorporated by reference in its entirety.

### TECHNICAL FIELD

This specification generally relates to predicting motor functions of a subject based on a biomarker.

### BACKGROUND

Nanobody® molecules are also known as heavy chain single variable domain (VHH) antibodies. Monoclonal and recombinant antibodies are important tools in medicine and biotechnology. Like all mammals, camelids (e.g., Ilamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain at their N-terminus called VHH (or Nanobody® molecule).

Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, or Nanobody® molecule) is the smallest antigen-binding domain generated by adaptive immune systems. The third Complementarity Determining Region (CDR3) of the variable region of these antibodies has been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into bispecific antibody constructs than constructs containing conventional VH-VL pairs or single domains based on VH domains.

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)2, scFv, di-scFv), wherein two immunoglobulin domains, in particular two variable domains, interact to form an antigen binding site.

Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation.

In contrast, immunoglobulin single variable domains are capable of specifically binding to an epitope of the antigen without pairing with an additional immunoglobulin variable domain. The binding site of an immunoglobulin single variable domain is formed by a single VH, a single VHH, or single VL domain. Hence, the antigen binding site of an immunoglobulin single variable domain is formed by no more than three CDRs.

As such, the single variable domain may be a light chain variable domain sequence (e.g., a VL-sequence) or a suitable fragment thereof, or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

A machine-learning model is a computational model that learns patterns and relationships in data, and then uses that knowledge to make predictions or decisions on new data. Neural networks are machine learning models that employ one or more layers of nonlinear units to predict an output for a received input. The output of each hidden layer is used as input to the next layer in the network, i.e., the next hidden layer or the output layer. Each layer of the network generates an output from a received input in accordance with current values of a respective set of parameters.

### SUMMARY

This disclosure describes methods, computer systems, and apparatus, including computer programs encoded on computer storage media, for predicting properties of Nanobody® molecules.

In this specification, the terms “VHH”, “Nanobody® molecules”, and “nanobody” are used synonymously.

In one aspect, this disclosure provides a prediction method for predicting one or more properties of a Nanobody® molecule. The method can be implemented by a system including one or more computers. The system obtains data representing an amino acid sequence of the Nanobody® molecule, generates an input vector by numerically encoding the amino acid sequence, and generates an embedded feature vector by processing the input vector using an embedding machine-learning model having a first set of model parameters. The first set of model parameters have been updated using self-supervised learning of a first machine-learning model that includes the embedding machine-learning model and is configured to perform a sequence reconstruction task. The system further processes the embedded feature vector using a property-prediction machine-learning model to generate an output that predicts one or more properties of the Nanobody@ molecule. The property-prediction machine-learning model has a second set of model parameters that have been updated using supervised learning, based on a plurality of training examples, of a second machine-learning model including the property-prediction machine-learning model. Each respective training example includes (i) a respective training input specifying a representation of a respective Nanobody® molecule and (ii) a respective label specifying one or more properties of the respective Nanobody® molecule.

In some implementations of the prediction method, the properties of the Nanobody® molecule can include a binding affinity to one or more target molecules, a binding specificity to one or more target molecules, a production yield, a stability under one or more environmental conditions, a cross-reactivity to one or more target molecules, a melting temperature, and/or an immunogenicity.

In some implementations of the prediction method, to generate the input vector, the system can map each amino acid of the amino acid sequence to a respective numerical value, and generate the token vector by combining (e.g., concatenating) the numerical values.

In some implementations of the prediction method, the first machine-learning model can include a large language model (LLM). For example, the first machine-learning can include: a variational autoencoder (VAE), an autoregressive transformer, and/or a bidirectional transformer.

In some implementations of the prediction method, the property-prediction machine-learning model can include a neural network, a K-nearest neighbors model, a support vector machine, a decision trees model, a random forest model, and/or a ridge regression model.

In some implementations of the prediction method, the first set of model parameters can be fixed after the self-supervised learning process and during the supervised learning process. In some other implementations of the prediction method, the first set of model parameters can be further updated during the supervised learning process wherein the embedding machine-learning model and the property-prediction machine-learning model are jointly trained end-to-end.

In some implementations of the prediction method, the system further maintains a first set of candidate machine-learning models that have been trained using self-supervised learning, and selects, from the first set of candidate machine-learning models, the embedding machine-learning model for a particular prediction task. To select the embedding machine-learning model from the first set of candidate machine-learning models, the system can evaluate a performance of each of the first set of candidate machine-learning models for the particular prediction task on a labeled dataset, and select the embedding machine-learning model from the first set of candidate machine-learning models based on the performances. The first set of candidate machine-learning models can includes two or more of: a variational autoencoder (VAE), an autoregressive transformer, or a bidirectional transformer.

In some implementations of the prediction method, based on the predicted properties of Nanobody® molecules, the system can select a Nanobody® molecule from a set of candidate Nanobody® molecules for performing a downstream task. The system can predict properties of each of the candidate Nanobody® molecules using the process described above, and select the Nanobody® molecule from the set of candidate Nanobody® molecules based on the predicted properties. The downstream task can include binding to a target protein, an agonist or antagonist function, achieving thermal stability, and/or achieving an oral stability.

In some implementations of the prediction method, the system further uses the embedding machine-learning model to generate a respective embedded feature vector for each of a plurality of Nanobody® molecules, and generates a prediction result by performing a clustering analysis on the embedded feature vectors of the plurality of Nanobody® molecules.

In some implementations of the prediction method, the system further maintains a dataset including, for each of a plurality of known Nanobody® molecules, data specifying (i) the amino acid sequence of the respective known Nanobody® molecule and (ii) respective embedded feature vector generated for the respective known Nanobody® molecule. The system receives an input specifying the amino acid sequence of a particular Nanobody® molecule, uses the embedding machine-learning model to generate a particular embedded feature vector for the particular Nanobody® molecule, searches the dataset to identify a set of one or more embedded feature vectors are within a predefined distance from the particular embedded feature vector in a feature space, and identifies amino acid sequences corresponding to the identified set of embedded feature vectors in the dataset; and outputting data specifying the identified amino acid sequences.

In some implementations of the prediction method, the property-prediction machine-learning model is configured to perform a regression task and/or a classification task.

In another aspect, this disclosure provides a design method for determining the optimal amino acid sequence of a Nanobody® molecule for performing a particular task. The design method can be implemented by a system including one or more computers. The system maintains data representing a set of candidate sequences for the Nanobody® molecule, and use a reinforcement-learning (RL) model to process one or more of the candidate sequences to generate one or more new sequences. The RL model has been trained using a reward signal including Nanobody® molecule properties predicted using the prediction method described above. The system can select an optimal sequence from the new sequences.

In another aspect, this disclosure provides a reinforcement-learning (RL) method for training an RL model for determining the optimal amino acid sequence of a Nanobody® molecule for performing a particular task. To perform the training, a computer-implemented system can use the RL model to process an input sequence representing a Nanobody® molecule to generate a set of one or more actions that modify the input sequence, determine a new sequence based on the input sequence and the set of actions, and compute one or more reward values indicative of how successfully the particular task is performed by a Nanobody® molecule represented by the new sequence. The reward values are computed using one or more Nanobody® molecule properties predicted using the prediction method described. The computer-implemented system can adjust one or more parameters of the RL model based on at least on the reward values.

In another aspect, this disclosure provides a training method for training a prediction model for predicting properties of Nanobody® molecules. The training method can be implemented by a system including one or more computers. The prediction model includes (i) an embedding machine-learning model configured to generate an embedded feature vector for a model input representing an amino acid sequence of the Nanobody® molecule and (ii) a property-prediction machine-learning model configured to process the embedded feature vector to generate an output specifying one or more properties of the Nanobody® molecule. The system obtains a first dataset including a set of sequence representations of Nanobody® molecules, performing self-supervised learning of a first machine-learning model including the embedding machine-learning model on a reconstruction task using the first data set, and obtains a second dataset including a plurality of training examples. Each respective training example includes (i) a respective training input specifying a representation of a respective Nanobody® molecule and (ii) a respective label specifying one or more properties of the respective Nanobody® molecule. The system performs supervised learning of a second machine-learning model including the property-prediction machine-learning on the second dataset.

In some implementations of the training method, the properties of the Nanobody® molecule include a binding affinity to a target molecule, a specificity, a yield, a cross-reactivity, a melting temperature, a stability, and/or an immunogenicity.

In some implementations of the training method, the first machine-learning model includes a large language model (LLM).

In some implementations of the training method, the first machine-learning model includes a variational autoencoder (VAE), an autoregressive transformer, and/or a bidirectional transformer.

In some implementations of the training method, the property-prediction machine-learning model comprises one or more of: a neural network, a K-nearest neighbors model, a support vector machine, a decision trees model, a random forest model, and/or a ridge regression model.

In some implementations of the training method, the first set of model parameters are fixed after the self-supervised learning process and during the supervised learning process.

In some other implementations of the training method, the first set of model parameters are further updated during the supervised learning process wherein the embedding machine-learning model and the property-prediction machine-learning model are jointly trained end-to-end.

This disclosure also provides a system including one or more computers and one or more storage devices storing instructions that, when executed by the one or more computers, cause the one or more computers to perform the prediction method, the training method, the design method, or the RL method described above.

This disclosure also provides one or more computer storage media storing instructions that when executed by one or more computers, cause the one or more computers to perform the prediction method, the training method, the design method, or the RL method described above.

In another aspect, this disclosure provides a second prediction method for training a prediction model for predicting properties of a heavy chain antibody single variable domain (ISVD) molecule, The prediction method can be implemented by a system including one or more computers. The system obtains a token sequence representing the ISVD molecule; generates an input vector by numerically encoding the token sequence representing the ISVD molecule, and generates an embedded feature vector by processing the input vector using an embedding machine-learning model having a first set of model parameters. The first set of model parameters have been updated using self-supervised learning of a first machine-learning model that comprises the embedding machine-learning model and is configured to perform a sequence reconstruction task. In some cases, the ISVD molecule is a VHH molecule.

The system processes the embedded feature vector using a property-prediction machine-learning model to generate an output that predicts one or more properties of the ISVD molecule. The property-prediction machine-learning model has a second set of model parameters that have been updated using supervised learning, based on a plurality of training examples, of a second machine-learning model comprising the property-prediction machine-learning model. Each respective training example comprises (i) a respective training input specifying a representation of a respective ISVD molecule and (ii) a respective label specifying one or more properties of the respective ISVD molecule.

In some implementations of the second prediction method, the ISVD molecule is a single variable domain of an Immunoglobulin G that (i) comprises two heavy chains and (ii) lacks any CHI domains.

In some implementations of the second prediction method, to obtain the token sequence representing the ISVD molecule, the system obtains an initial token sequence that represents an amino acid sequence of the ISVD molecule; and generates the token sequence with a predefined length by appending a padding token at one or more positions to the initial token sequence. The predefined length is greater than the length of the initial token sequence.

In some implementations of the second prediction method, to obtain the token sequence representing the ISVD molecule, the system obtains an initial token sequence that represents an amino acid sequence of the ISVD molecule; and generates the token sequence by performing an alignment of the initial token sequence. The alignment comprises inserting a gap token at one or more positions in the initial token sequence. In some cases, the alignment is performed according to annotation information of the initial token sequence. In some cases, the annotation information of the initial token sequence is generated using an annotation scheme selected from the IMGT annotation scheme, the Kabat annotation scheme, the Chothia annotation scheme, the Martin annotation scheme, the Wolfguy annotation scheme, or the AHo annotation schemes annotation scheme. In a particular example, the annotation information of the initial token sequence is generated using the AHo annotation schemes annotation scheme.

In some implementations of the second prediction method, the one or more properties of the ISVD molecule comprises one or more of: a binding affinity to one or more target molecules, a binding specificity to one or more target molecules, a production yield, a stability under one or more environmental conditions, a cross-reactivity to one or more target molecules, a melting temperature, or an immunogenicity.

In some implementations of the second prediction method, to generate the input vector, the system maps each token in the token sequence to a respective numerical value; and generates the input vector by concatenating the numerical values.

In some implementations of the second prediction method, the first machine-learning model comprises a large language model (LLM).

In some implementations of the second prediction method, the first machine-learning comprises: a variational autoencoder (VAE), an autoregressive transformer, or a bidirectional transformer.

In some implementations of the second prediction method, the property-prediction machine-learning model comprises one or more of: a neural network, a K-nearest neighbors model, a support vector machine, a decision trees model, a random forest model, or a ridge regression model.

In some implementations of the second prediction method, the first set of model parameters are fixed after the self-supervised learning process and during the supervised learning process.

In some implementations of the second prediction method, the first set of model parameters are further updated during the supervised learning process wherein the embedding machine-learning model and the property-prediction machine-learning model are jointly trained end-to-end.

In some implementations of the second prediction method, the system further maintains a first set of candidate machine-learning models that have been trained using self-supervised learning; and selects, from the first set of candidate machine-learning models, the embedding machine-learning model for a particular prediction task.

In some implementations of the second prediction method, to select the embedding machine-learning model from the first set of candidate machine-learning models, the system evaluates a performance of each of the first set of candidate machine-learning models for the particular prediction task on a labeled dataset; and selects the embedding machine-learning model from the first set of candidate machine-learning models based on the performances.

In some implementations of the second prediction method, wherein the first set of candidate machine-learning models includes two or more of: a variational autoencoder (VAE), an autoregressive transformer, or a bidirectional transformer.

In some implementations of the second prediction method, the system further uses the embedding machine-learning model to generate a respective embedded feature vector for each of a plurality of ISVD molecules; and generates a prediction result by performing a clustering analysis on the embedded feature vectors of the plurality of ISVD molecules.

In some implementations of the second prediction method, the system further maintains a dataset comprising, for each of a plurality of known ISVD molecules, data specifying (i) the amino acid sequence of the respective known ISVD molecule and (ii) respective embedded feature vector generated for the respective known ISVD molecule. The system receives an input specifying the amino acid sequence of a particular ISVD molecule; uses the embedding machine-learning model to generate a particular embedded feature vector for the particular ISVD molecule; searches the dataset to identify a set of one or more embedded feature vectors are within a predefined distance from the particular embedded feature vector in a feature space; identifies amino acid sequences corresponding to the identified set of embedded feature vectors in the dataset; and outputs data specifying the identified amino acid sequences.

In some implementations of the second prediction method, the property-prediction machine-learning model is configured to perform one or more of a regression task or a classification task.

In another aspect, this disclosure provides a second selection method for selecting an ISVD molecule from a set of candidate ISVD molecules for performing a downstream task.

The selection method can be implemented by a system including one or more computers. The system predicts properties of each of the candidate ISVD molecules using the method according to any one of the preceding methods; and selects the ISVD molecule from the set of candidate ISVD molecules based on the predicted properties.

In some implementations of the second selection method, the downstream task includes one or more of: binding to a target protein, an agonist or antagonist function, achieving thermal stability, or achieving an oral stability.

In another aspect, this disclosure provides a second design method for determining an optimal amino acid sequence of an ISVD molecule for performing a particular task. The design method can be implemented by a system including one or more computers. The system maintains data representing a set of candidate sequences for the ISVD molecule; and uses a reinforcement-learning (RL) model to process one or more of the candidate sequences to generate one or more new sequences. The RL model has been trained using a reward signal comprising ISVD molecule properties predicted using the method of any of the preceding claims. The system selects an optimal sequence from the new sequences.

In another aspect, this disclosure provides a second RL method for training a reinforcement-learning (RL) model for determining an optimal amino acid sequence of an ISVD molecule for performing a particular task. The training method can be implemented by a system including one or more computers. The system uses the RL model to process an input sequence representing an ISVD molecule to generate a set of one or more actions that modify the input sequence; determines a new sequence based on the input sequence and the set of actions; and computes one or more reward values indicative of how successfully the particular task is performed by an ISVD molecule represented by the new sequence. The reward values are computed using one or more ISVD molecule properties predicted using the method of any of the preceding claims. The system adjusts one or more parameters of the RL model based at least on the reward values.

In another aspect, this disclosure provides a second training method for training a prediction model for predicting properties for an ISVD molecule. The training method can be implemented by a system including one or more computers. The prediction model includes (i) an embedding machine-learning model configured to generate an embedded feature vector for a model input representing the ISVD molecule and (ii) a property-prediction machine-learning model configured to process the embedded feature vector to generate an output specifying one or more properties of the ISVD molecule. The system obtains a first dataset comprising a set of sequence representations of ISVD molecules; performs self-supervised learning of a first machine-learning model comprising the embedding machine-learning model on a reconstruction task using the first data set; obtains a second dataset comprising a plurality of training examples, each respective training example comprising (i) a respective training input specifying a representation of a respective ISVD molecule and (ii) a respective label specifying one or more properties of the respective ISVD molecule; and performs supervised learning of a second machine-learning model comprising the property-prediction machine-learning on the second dataset.

In some implementations of the second training method, the one or more properties of the ISVD molecule comprises one or more of: a binding affinity to a target molecule, a specificity, a yield, a cross-reactivity, a melting temperature, a stability, or an immunogenicity.

In some implementations of the second training method, the first machine-learning model comprises a large language model (LLM).

In some implementations of the second training method, the first machine-learning model comprises: a variational autoencoder (VAE), an autoregressive transformer, or a bidirectional transformer.

In some implementations of the second training method, the property-prediction machine-learning model comprises one or more of: a neural network, a K-nearest neighbors model, a support vector machine, a decision trees model, a random forest model, or a ridge regression model.

In some implementations of the second training method, the first set of model parameters are fixed after the self-supervised learning process and during the supervised learning process.

In some implementations of the second training method, the first set of model parameters are further updated during the supervised learning process wherein the embedding machine-learning model and the property-prediction machine-learning model are jointly trained end-to-end.

This disclosure also provides a system including one or more computers and one or more storage devices storing instructions that, when executed by the one or more computers, cause the one or more computers to perform the second prediction method, the second training method, the second design method, or the second RL method described above.

This disclosure also provides one or more computer storage media storing instructions that when executed by one or more computers, cause the one or more computers to perform the second prediction method, the second training method, the second design method, or the second RL method described above.

The subject matter described in this disclosure can be implemented in particular embodiments so as to realize one or more advantages.

The goals of Nanobody® molecule engineering are to design, create, and/or select Nanobody® molecules with optimized properties for specific applications. One approach of Nanobody® molecule engineering includes generating a large number of candidate Nanobody® molecules, measuring the fitness of each Nanobody® molecule for the specific application, and selecting the fittest Nanobody® molecule. This process can be iteratively performed by diversifying the selected Nanobody® molecule to generate the candidate Nanobody® molecules for the next iteration.

Existing Nanobody® molecule engineering processes are associated with many challenges, such as multi-objective optimization (i.e., the need to optimize multiple properties for a specific application) and a large search space. For example, for a 14 amino acid CDRH3, the sequence space is as large as 1.6×10¹⁸. Experimental benchmarking such a search space becomes unattainable.

The described techniques use deep learning to computationally predict Nanobody® molecule properties based on the amino acid sequence of the Nanobody® molecule. In particular, the described techniques use self-supervised learning to pre-train language models for generating effective embeddings for Nanobody® molecule sequences, followed by transferred learning using labeled data for downstream tasks. The self-supervised pre-training process makes it possible to generate high-performance embeddings when labeled data is limited for downstream tasks.

Based on the predicted Nanobody® molecule properties, the described system or another system can determine optimal Nanobody® molecule sequences for performing specific applications. For example, the system can generate an output that indicates whether a particular Nanobody® molecule is suitable for a particular application, an output that specifies the optimal Nanobody® molecule sequence for the particular application, or an output that indicates where mutations can be made in the sequence. The system can transmit the output to a fabrication apparatus operative to implement the instruction to produce the Nanobody® molecule. Overall, by training high-performance prediction models based on limited experimental data and using the trained model to predict Nanobody® molecule properties, the described techniques can greatly improve the efficacy and efficiency of Nanobody® molecule engineering.

The details of one or more embodiments of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example of a Nanobody® molecule-property prediction system.

FIG. 2 is a flow diagram illustrating an example process for predicting properties of a Nanobody® molecule.

FIG. 3 is a flow diagram illustrating an example process for training a prediction model for predicting properties of Nanobody® molecules.

FIG. 4 is a block diagram of an example computer system.

Like reference numbers and designations in the various drawings indicate like elements.

### DETAILED DESCRIPTION

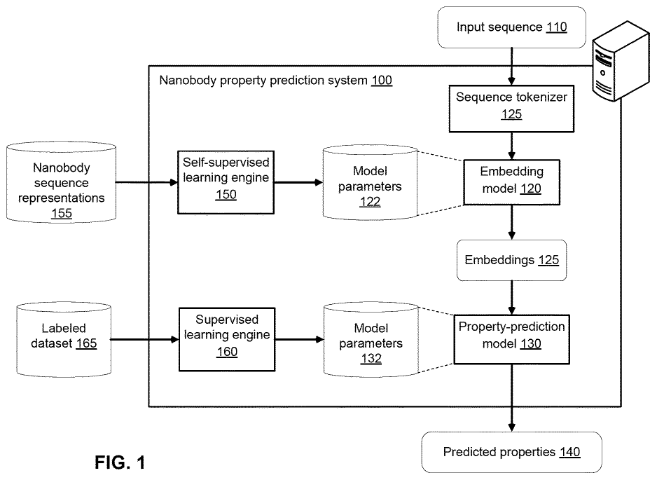

FIG. 1 shows an example of a Nanobody® molecule-property prediction system 100. The system 100 is an example of a system implemented as computer programs on one or more computers in one or more locations, in which the systems, components, and techniques described below can be implemented.

The Nanobody® molecule-property prediction system 100 uses a machine-learning model to predict one or more properties 140 of a Nanobody® molecule based on input data 110 specifying the amino acid sequence of the Nanobody® molecule. In some embodiments, the entire Nanobody® molecule sequence is used. In some embodiments, the three CDR sequences (CDR1, CDR2, and CDR3) are used. In some embodiments, only the CDR3 sequence is used.

In general, the machine-learning model includes an embedding machine-learning model 120 having a first set of model parameters 122 and a property-prediction machine-learning model 130 having a second set of model parameters 132.

The system 100 includes a sequence tokenizer 125 configured to generate an input vector as an input to the embedding machine-learning model 120. The sequence tokenizer 125 generates the input vector by numerically encoding a token sequence that represents the Nanobody® molecule. For example, the system can generate the input vector by concatenating numerical values assigned to different tokens in the token sequence.

In some implementations, the token sequence for generating the input vector corresponds to the amino sequence of the Nanobody® molecule. That is, the input vector is generated by numerically encoding the raw amino acid sequence of the Nanobody® molecule. In some cases, to ensure a fixed length for the input vector, the amino acid sequence of the Nanobody® molecule may be appended with a padding token at the end positions, extending the token sequence to a predefined length, for example, 200 tokens.

In some implementations, the amino acid sequence of the Nanobody® molecule is aligned to generate the token sequence representing the Nanobody® molecule. For example, the sequence tokenizer 125 can align each amino acid sequence by inserting a gap token at certain positions in the respective amino acid sequence to match the positions of identical or similar regions, e.g., the framework regions and CDRs, across multiple sequences. In some cases, the alignment can be performed based on structural and/or functional annotations of the amino acid sequence of the Nanobody® molecule. Any appropriate annotation scheme can be used to generate the annotations. Examples of annotation schemes include the IMGT, Kabat, Chothia, Martin, Wolfguy, and AHo annotation schemes. In one particular example, the alignment can be performed based on annotations obtained using the AHo annotation scheme which provides hydrophobicity information of the amino acids. The length of the aligned token sequence can depend on the annotation scheme. In an illustrative example, the aligned token sequence has a length of 151. The sequence tokenizer 125 can generate the input vector by numerically encoding the aligned token sequence.

Using the aligned token sequence of the Nanobody® molecule to generate the input vector provides several advantages. Mapping amino acids to their functional and/or structural positions within the Nanobody® molecule facilitates the machine-learning model to establish clearer relationships between sequence elements. Pre-aligned sequences can help the model focus on meaningful patterns, e.g., the aligned framework regions and CDRs, rather than being distracted by the uncertainty of the positions of the regions. In addition, a shorter token sequence (e.g., 151 vs. 200) can potentially improve the speed of training the model.

The embedding machine-learning model 120 is configured to process the input vector to generate an embedded feature vector 125. The embedded feature vector 125 is a numerical representation of input data that captures the essential information required for one or more tasks. In particular, the embedded feature vector 125 can be a high-dimensional vector of real numbers that captures features of the model input specifying the Nanobody® molecule. These could be amino acid level embeddings or embeddings at the level of the protein (global embeddings).

In some implementations, the embedding machine-learning model 120 is a neural network. The embedding neural network can adopt any appropriate architecture. In particular, the embedding neural network 120 can include at least a portion (e.g., the embedding portion) of a state-of-the-art language model.

For example, in some implementations, the embedding neural network 120 can include the encoder network of a variational autoencoder (VAE). Implementation examples of a VAE are described in “Auto-encoding variational Bayes,” Kingma et al., arXiv:1312.6114, 2013.

In some implementations, the embedding neural network 120 can include the embedding layers of an autoregressive transformer, e.g., a generative pre-trained transformer (GPT). Implementation examples of the GPT are described in “Language Models are Few-Shot Learners,” Brown et al., Advances in Neural Information Processing Systems 33:1877-1901, 2020.

In some implementations, the embedding neural network 120 can include a bidirectional transformer, e.g., a bidirectional encoder representations from transformers (BERT) model. Implementation examples of the BERT are described in “BERT: Pre-training of Deep Bidirectional Transformers for Language Understanding,” Devlin et al., arXiv:1810.04805, 2018.

Using a language model to generate embeddings for a Nanobody® molecule sequence provides several advantages for predicting Nanobody® molecule functions and properties in the downstream task. Due to evolutionary pressures, Nanobody® molecule sequences are not random. For example, a Nanobody® molecule can include evolutionarily conserved regions and portions of the Nanobody® molecule sequence can be reused. Further, correlations and interactions can exist between pairs of positions in a Nanobody® molecule. Language models can learn complex patterns in Nanobody® molecule sequences, and can be used to identify previously unknown patterns and correlations in Nanobody® molecule sequences. As will be described in more detail below, the language models for generating the Nanobody® molecule embeddings are trained specifically using Nanobody® molecule sequence data. This is important for obtaining a high-performance model for Nanobody® molecule property prediction, that is, for obtaining a model having high prediction accuracy and avoiding model bias or overfitting.

The system 100 further includes a property-prediction machine-learning model 130 configured to process the embedded feature vector 125 to generate an output 140 that predicts one or more properties of the Nanobody® molecule. The predicted properties can be Nanobody® molecule properties that are relevant to one or more applications, and can include one or more of: a binding affinity to one or more target molecules, a binding specificity to one or more target molecules, a production yield, a stability under one or more environmental conditions, a cross-reactivity to one or more target molecules, a melting temperature, and/or an immunogenicity of the Nanobody® molecule. In an illustrative example, the intended application is to screen the suitability of a Nanobody® molecule to be used as a treatment agent via oral delivery, and the properties of interest would include the oral stability of the Nanobody® molecule.

The property-prediction machine-learning model 130 can adopt any suitable machine-learning techniques, and can include one or more of: a neural network, a K-nearest neighbors model, a support vector machine, a decision trees model, a random forest model, or a ridge regression model. The property-prediction machine-learning model 130 can be used to perform a regression task, a classification task, or both.

In some implementations, the system 100 or another system includes a self-supervised learning engine 150 configured to update the model parameters 122 of the embedding machine-learning model using self-supervised learning, based on a set of Nanobody® molecule sequence representations 155. The goal of the self-supervised learning is to learn meaningful embeddings of Nanobody® molecule sequences without needing to use labeled data. Instead, the self-supervised learning engine 150 learns the embeddings using unlabeled Nanobody® molecule sequence data, that is, data specifying or representing each of a set of Nanobody® molecule sequences without Nanobody® molecule property labels. Thus, the self-supervised learning engine 150 can leverage the large number of known Nanobody® molecule sequences to learn the embeddings without needing to obtain a large amount of experimental benchmark data for the properties of the known Nanobody® molecules.

In order to effectively learn the embeddings from unlabeled data, the self-supervised learning engine 150 can be configured to train a first machine-learning model to perform a reconstruction task, that is, a task for generating embeddings for an input Nanobody® molecule sequence representation, and reconstructing the input Nanobody® molecule sequence representation from the embeddings. The first machine-learning model includes the embedding machine-learning model 120 as a sub-network for generating the embeddings.

The self-supervised learning engine 150 can update the parameters of the first machine-learning model (including the model parameters 122 of the embedding machine-learning model 120) by minimizing a reconstruction error between the input Nanobody® molecule sequence and the reconstructed Nanobody® molecule sequence. The self-supervised learning engine 150 can update the model parameters using any appropriate backpropagation-based machine learning technique, e.g., using the Adam or AdaGrad optimizers.

In some implementations, after the self-supervised learning, the embedding machine-learning model 120 can be used for different prediction tasks, i.e., tasks for predicting different combinations of Nanobody® molecule properties. That is, the embedding machine-learning model 120 may not need to be re-trained for predicting different Nanobody® molecule properties.

The system 100 or another system can further include a supervised learning engine 160 configured to update the model parameters 132 of the property-prediction model 130 based on a labeled dataset 165. The labeled dataset 165 includes a plurality of labeled training examples. Each training example includes (i) a training input specifying a representation of a respective Nanobody® molecule and (ii) a label specifying one or more properties of the respective Nanobody® molecule. The Nanobody® molecule labels can be based on experimental measurements of the properties of the corresponding Nanobody® molecules.

The supervised learning engine 160 is configured to perform supervised learning of a second machine-learning model including the property-prediction machine-learning model 130 on the labeled dataset 165. That is, the supervised learning engine 160 is configured to update the parameters of the second machine-learning model (including the model parameters 132 of the property-prediction machine-learning model 130) based on the labeled dataset 165.

In some implementations, the second machine-learning model further includes the embedding machine-learning model 120. That is, the model parameters 122 of the embedding machine-learning model 120 are further fine-tuned end-to-end with the property-prediction machine-learning model 130 via supervised learning based on the labeled dataset 165.

In some other implementations, the model parameters 122 of the embedding machine-learning model 120 are fixed during the supervised learning when the model parameters 132 of the property-prediction machine-learning model 130 are being updated.

The supervised learning engine 160 can update the parameters of the second machine-learning model (including model parameters 132 of the property-prediction machine-learning model 130, and optionally including the model parameters 122 of the embedding machine-learning model 120) by minimizing a prediction error between the predicted Nanobody® molecule properties and the properties specified in the labels. The supervised learning engine 160 can update the model parameters using any appropriate backpropagation-based machine learning technique, e.g., using the Adam or AdaGrad optimizers.

In some implementations, the system 100 or another system can maintain a set of candidate embedding machine-learning models that have been trained using self-supervised learning. For example, the system 100 can maintain embedding machine-learning models with different network architectures, including, for example, the variational autoencoder (VAE), the autoregressive transformer, and the bidirectional transformer. The system 100 can select the embedding machine-learning model 120 from the set of pre-trained candidate embedding machine-learning models for a particular prediction task, e.g., by evaluating the performance of each candidate embedding machine-learning model for the particular prediction task on a labeled dataset and selecting the best-performing embedding machine-learning model from the first set of candidate machine-learning models. Here, a particular prediction task can be a task for predicting a particular set of Nanobody® molecule properties that are relevant to a specific application. To evaluate the performance of a pre-trained candidate embedding machine-learning model for the particular prediction task, the system can pair the pre-trained candidate embedding machine-learning model with a property-prediction model 130 for the particular prediction task, and evaluate the performance, e.g., prediction accuracy, of the paired models for the prediction task.

In some implementations, the system 100 can mix and match different candidate embedding machine-learning models with different types of property-prediction models (e.g., neural networks, K-nearest neighbors models, support vector machines, decision trees models, random forest models, or ridge regression models) and select the best-performing combination for a particular prediction task. The system 100 can use any appropriate optimization method for identifying the best-performing combination of the embedding machine-learning model and the property-prediction model.

Based on the predicted Nanobody® molecule properties 140, the system 100 can determine optimal Nanobody® molecule sequences for performing specific downstream tasks, e.g., binding to a target protein, an agonist or antagonist function, achieving thermal stability, and/or achieving an oral stability. For example, the system can generate an output that indicates whether a particular Nanobody® molecule is suitable for a particular application, or an output that specifies the optimal Nanobody® molecule sequence for the particular application. The system can transmit the output to a fabrication apparatus operative to implement the instruction to produce the Nanobody® molecule.

In some implementations, the system 100 or another system can utilize the embedded feature vectors 125 generated for multiple Nanobody® molecules to perform clustering analysis. The clustering analysis involves grouping Nanobody® molecules based on the distances between pairs of corresponding embedded feature vectors 125 of the Nanobody® molecules in the embedded feature space. The results of the clustering analysis can provide insight into common properties among different Nanobody® molecule sequences. These insights can be used to select and/or design Nanobody® molecule sequences with properties suitable for particular applications.

In some implementations, the system 100 or another system can perform search of Nanobody® molecule sequences in the embedded feature vector space to identify Nanobody® molecules that have certain properties similar to a particular Nanobody® molecule. The system can maintain a database of sequences and corresponding embedded feature vectors for a population of Nanobody® molecules. When a search inquiry specifies the amino acid sequence of a particular Nanobody® molecule, the system can use the embedding machine-learning model 120 to generate a particular embedded feature vector for the particular Nanobody® molecule. The system can then search the database for a set of embedded feature vectors that are within a predefined distance from the particular embedded feature vector in the embedded feature space and identify the Nanobody® molecules corresponding to the identified set of embedded feature vectors in the database. The resulting Nanobody® molecule sequences can be used to guide the selection and/or design of Nanobody® molecule sequences with properties suitable for particular applications.

In some implementations, the system 100 or another system can generate an output that specifies the optimal Nanobody® molecule sequence for a particular application using reinforcement learning based on the predicted Nanobody® molecule properties 140. For example, the system 100 can train a reinforcement-learning (RL) model for determining the optimal amino acid sequence of a Nanobody® molecule for performing the particular task.

The system 100 can use any appropriate reinforcement learning technique for training the RL model. In general, the RL model is configured to process an input sequence representing a Nanobody® molecule to generate a set of one or more actions that modify the input sequence. The system 100 can determine new sequences based on the output of the RL model. The system 100 can compute one or more reward values indicative of how successfully the particular task is performed by a Nanobody® molecule represented by the new sequence. The reward values are computed using one or more Nanobody® molecule properties predicted using the embedding machine-learning model 120 and the property-prediction model 130. The system can adjust the parameters of the RL model based at least on the reward values.

After the RL model has been trained, the system 100 or another system can use the RL model to generate Nanobody® molecule sequences for target properties. In a particular example, the system 100 can maintain a curriculum of Nanobody® molecule sequences, i.e., data representing a set of candidate sequences for the Nanobody® molecule. The system can use the trained RL model to process the candidate sequences to generate new sequences and select one or more optimal sequences from the new sequences. The system 100 can repeatedly perform the process to iteratively update the curriculum of Nanobody® molecule sequences until a condition is reached, e.g., when a Nanobody® molecule sequence has been determined to have a satisfactory performance or a threshold number of iterations has been reached.

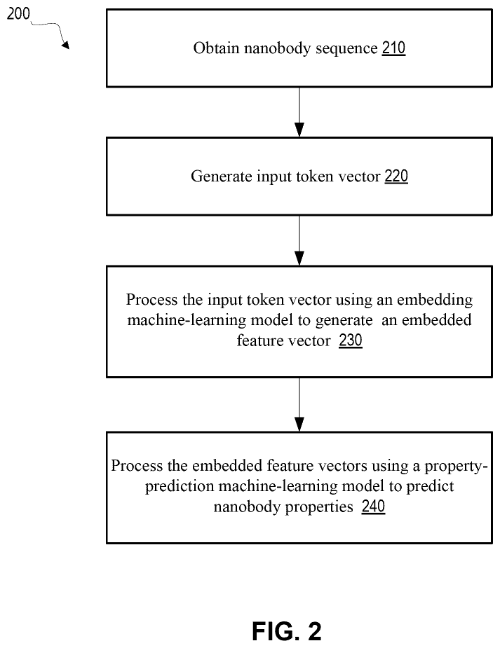

FIG. 2 is a flow diagram illustrating an example process 200 for predicting properties of a Nanobody® molecule. For convenience, the process 200 will be described as being performed by a system of one or more computers located in one or more locations. For example, a Nanobody® molecule-property prediction system, e.g., the Nanobody® molecule-property prediction system 100 of FIG. 1, appropriately programmed in accordance with this disclosure, can perform the process 200.

At 210, the system obtains a Nanobody® molecule sequence, i.e., a token sequence representing the Nanobody® molecule. In some implementations, the token sequence can be an aligned sequence generated by aligning the amino acid sequence of the Nanobody® molecule.

At 220, the system generates an input vector by numerically encoding the token sequence. For example, the system can generate a vector by concatenating numerical values assigned to different amino acids to form the input vector.

At 230, the system generates an embedded feature vector by processing the input vector using an embedding machine-learning model. The embedding machine-learning model having a first set of model parameters. The first set of model parameters have been updated using self-supervised learning of a first machine-learning model that includes the embedding machine-learning model and configured to perform a sequence reconstruction task.

At 240, the system processes the embedded feature vector using a property-prediction machine-learning model to generate an output that predicts one or more properties of the Nanobody® molecule, including, for example, a binding affinity to one or more target molecules, a binding specificity to one or more target molecules, a production yield, a stability under one or more environmental conditions; a cross-reactivity to one or more target molecules, a melting temperature, and/or an immunogenicity of the Nanobody® molecule.

The property-prediction machine-learning model has a second set of model parameters. The second set of model parameters have been updated using supervised learning, based on a plurality of training examples, of a second machine-learning model including the property-prediction machine-learning model.

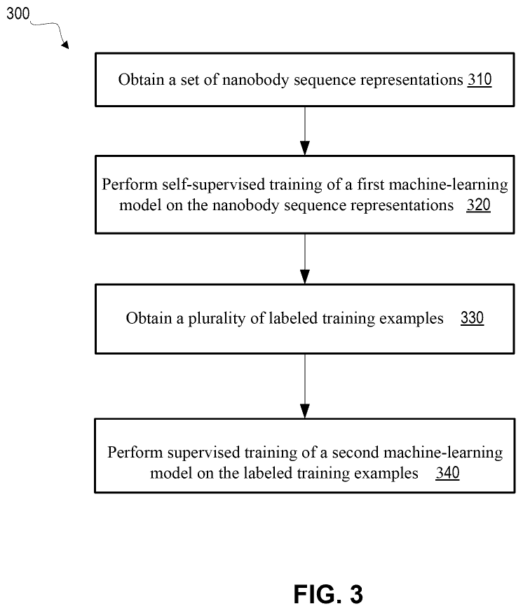

FIG. 3 is a flow diagram illustrating an example process 300 for training a prediction model for predicting properties of Nanobody® molecules. For convenience, the process 300 will be described as being performed by a system of one or more computers located in one or more locations. For example, a Nanobody® molecule-property prediction system, e.g., the Nanobody® molecule-property prediction system 100 of FIG. 1, appropriately programmed in accordance with this disclosure, can perform the process 300.

In general, the prediction model includes (i) an embedding machine-learning model configured to generate an embedded feature vector for a model input representing an amino acid sequence of the Nanobody® molecule and (ii) a property-prediction machine-learning model configured to process the embedded feature vector to generate an output specifying one or more properties of the Nanobody® molecule.

At 310, the system obtains a first dataset including a set of sequence representations of Nanobody® molecules. For example, the sequence representation can be a token vector that numerically encodes the sample sequence of a Nanobody® molecule for which the amino acid sequence is known.

At 320, the system performs self-supervised learning of a first machine-learning model including the embedding machine-learning model on a reconstruction task using the first data set. The first machine-learning model can be a large language model and can include a variational autoencoder (VAE), an autoregressive transformer, or a bidirectional transformer.

At 330, the system obtains a second dataset including a plurality of training examples. Each training example includes (i) a respective training input specifying a representation of a respective Nanobody® molecule and (ii) a respective label specifying one or more properties of the respective Nanobody® molecule.

At 340, the system performs supervised learning of a second machine-learning model including the property-prediction machine-learning model based on the second dataset. The second machine-learning model can include one or more of: a neural network, a K-nearest neighbors model, a support vector machine, a decision trees model, a random forest model, or a ridge regression model. The second machine-learning model can be used to perform a regression task, a classification task, or both.

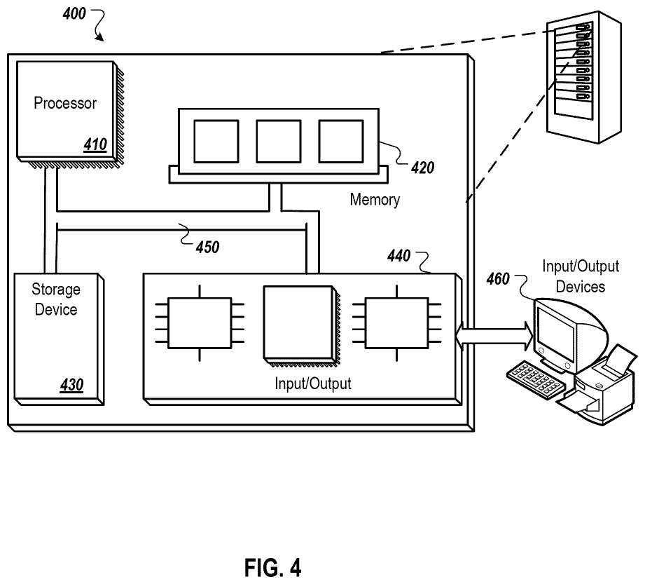

FIG. 4 is a block diagram of an example computer system 400 that can be used to perform operations described above. The system 400 includes a processor 410, a memory 420, a storage device 430, and an input/output device 440. Each of the components 410, 420, 430, and 440 can be interconnected, for example, using a system bus 450. The processor 410 is capable of processing instructions for execution within the system 400. In one implementation, the processor 410 is a single-threaded processor. In another implementation, the processor 410 is a multi-threaded processor. The processor 410 is capable of processing instructions stored in the memory 420 or on the storage device 430.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 can include, for example, a hard disk device, an optical disk device, a storage device that is shared over a network by multiple computing devices (for example, a cloud storage device), or some other large capacity storage device.

The input/output device 440 provides input/output operations for the system 400. In one implementation, the input/output device 440 can include one or more network interface devices, for example, an Ethernet card, a serial communication device, for example, a RS-232 port, and/or a wireless interface device, for example, a 502.11 card. In another implementation, the input/output device can include driver devices configured to receive data and send output data to other input/output devices, for example, keyboard, printer and display devices 460. Other implementations, however, can also be used, such as mobile computing devices, mobile communication devices, set-top box television client devices, etc.

Although an example processing system has been described in FIG. 4, implementations of the subject matter and the functional operations described in this disclosure can be implemented in other types of digital electronic circuitry, or in computer software, firmware, or hardware, including the structures disclosed in this disclosure and their structural equivalents, or in combinations of one or more of them.

This disclosure uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions. Embodiments of the subject matter and the functional operations described in this disclosure can be implemented in digital electronic circuitry, in tangibly-embodied computer software or firmware, in computer hardware, including the structures disclosed in this disclosure and their structural equivalents, or in combinations of one or more of them. Embodiments of the subject matter described in this disclosure can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this disclosure, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this disclosure the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this disclosure can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework,. e.g., a PyTorch or a TensorFlow framework.

Embodiments of the subject matter described in this disclosure can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this disclosure, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this disclosure contains many specific implementation details, these should not be construed as limitations on the scope of any invention or on the scope of what may be claimed, but rather as descriptions of features that may be specific to particular embodiments of particular inventions. Certain features that are described in this disclosure in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Furthermore, experiments can be performed to determine the predicted properties of the Nanobody® molecules, e.g., a binding affinity to a target molecule, a specificity, a yield, a cross-reactivity, a melting temperature, a stability, or an immunogenicity. In some embodiments, the experiment results can be used to adjust the methods as described herein. In some embodiments, the amino acid sequences of Nanobody® molecules with desired properties can be determined or predicted by the methods described herein. Recombinant vectors (e.g., expression vectors) that include an isolated polynucleotide (e.g., a polynucleotide that encodes the desired Nanobody® molecule sequence) can be used to express the Nanobody® molecule. In some embodiments, an expression vector is used. The polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector. The vector can be introduced into the host cell by methods known in the art, e.g., electroporation, chemical transfection (e.g., DEAE-dextran), transformation, transfection, and infection and/or transduction (e.g., with recombinant virus). Non-limiting examples of vectors include viral vectors (which can be used to generate recombinant virus), naked DNA or RNA, plasmids, cosmids, phage vectors, and DNA or RNA expression vectors associated with cationic condensing agents.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A computer-implemented method for predicting properties of an immunoglobulin single variable domains (ISVD) molecule, the method comprising:
obtaining a token sequence representing the ISVD molecule;
generating an input vector by numerically encoding the token sequence representing the ISVD molecule;
generating an embedded feature vector by processing the input vector using an embedding machine-learning model having a first set of model parameters, wherein the first set of model parameters have been updated using self-supervised learning of a first machine-learning model that comprises the embedding machine-learning model and is configured to perform a sequence reconstruction task; and
processing the embedded feature vector using a property-prediction machine-learning model to generate an output that predicts one or more properties of the ISVD molecule, wherein the property-prediction machine-learning model has a second set of model parameters that have been updated using supervised learning, based on a plurality of training examples, of a second machine-learning model comprising the property-prediction machine-learning model, each respective training example comprising (i) a respective training input specifying a representation of a respective ISVD molecule and (ii) a respective label specifying one or more properties of the respective ISVD molecule.

2. The method of claim 1, wherein the ISVD molecule a heavy chain antibody single variable domain (VHH) molecule.

3. The method of claim 1, wherein the ISVD molecule is a single variable domain of an Immunoglobulin G that (i) comprises two heavy chains and (ii) lacks any CH1 domains.

4. The method of claim 1, wherein obtaining the token sequence representing the ISVD molecule comprises:
obtaining an initial token sequence that represents an amino acid sequence of the ISVD molecule; and
generating the token sequence with a predefined length by appending a padding token at one or more positions to the initial token sequence, wherein the predefined length is greater than the length of the initial token sequence.

5. The method of claim 1, wherein obtaining the token sequence representing the ISVD molecule comprises:
obtaining an initial token sequence that represents an amino acid sequence of the ISVD molecule; and
generating the token sequence by performing an alignment of the initial token sequence, the alignment comprises inserting a gap token at one or more positions in the initial token sequence.

6. The method of claim 5, wherein the alignment is performed according to annotation information of the initial token sequence.

7. The method of claim 6, wherein the annotation information of the initial token sequence is generated using an annotation scheme selected from the IMGT annotation scheme, the Kabat annotation scheme, the Chothia annotation scheme, the Martin annotation scheme, the Wolfguy annotation scheme, or the AHo annotation schemes annotation scheme.

8. The method of claim 6, wherein the annotation information of the initial token sequence is generated using the AHo annotation schemes annotation scheme.

9. The method of claim 1, wherein the one or more properties of the ISVD molecule comprises one or more of: a binding affinity to one or more target molecules, a binding specificity to one or more target molecules, a production yield, a stability under one or more environmental conditions, a cross-reactivity to one or more target molecules, a melting temperature, or an immunogenicity.

10. The method of claim 1, wherein generating the input vector comprises:
mapping each token in the token sequence to a respective numerical value; and
generating the input vector by concatenating the numerical values.

11. The method of claim 1, wherein the first machine-learning model comprises a large language model (LLM).

12. The method of claim 1, wherein the first machine-learning comprises: a variational autoencoder (VAE), an autoregressive transformer, or a bidirectional transformer.

13. The method of claim 1, wherein the property-prediction machine-learning model comprises one or more of: a neural network, a K-nearest neighbors model, a support vector machine, a decision trees model, a random forest model, or a ridge regression model.

14. The method of claim 1, wherein the first set of model parameters are fixed after the self-supervised learning process and during the supervised learning process.

15. The method of claim 1, wherein the first set of model parameters are further updated during the supervised learning process wherein the embedding machine-learning model and the property-prediction machine-learning model are jointly trained end-to-end.

16. The method of claim 1, further comprising:
maintaining a first set of candidate machine-learning models that have been trained using self-supervised learning; and
selecting, from the first set of candidate machine-learning models, the embedding machine-learning model for a particular prediction task.

17. The method of claim 16, wherein selecting the embedding machine-learning model from the first set of candidate machine-learning models comprises:
evaluating a performance of each of the first set of candidate machine-learning models for the particular prediction task on a labeled dataset; and
selecting the embedding machine-learning model from the first set of candidate machine-learning models based on the performances.

18. The method of claim 16, wherein the first set of candidate machine-learning models includes two or more of: a variational autoencoder (VAE), an autoregressive transformer, or a bidirectional transformer.

19.-32 (canceled)

33. A system comprising:
one or more computers; and
one or more storage devices storing instructions that when executed by the one or more computers, cause the one or more computers to perform operations for predicting properties of an immunoglobulin single variable domains (ISVD) molecule, the operations comprising:
obtaining a token sequence representing the ISVD molecule;
generating an input vector by numerically encoding the token sequence representing the ISVD molecule;
generating an embedded feature vector by processing the input vector using an embedding machine-learning model having a first set of model parameters, wherein the first set of model parameters have been updated using self-supervised learning of a first machine-learning model that comprises the embedding machine-learning model and is configured to perform a sequence reconstruction task; and
processing the embedded feature vector using a property-prediction machine-learning model to generate an output that predicts one or more properties of the ISVD molecule, wherein the property-prediction machine-learning model has a second set of model parameters that have been updated using supervised learning, based on a plurality of training examples, of a second machine-learning model comprising the property-prediction machine-learning model, each respective training example comprising (i) a respective training input specifying a representation of a respective ISVD molecule and (ii) a respective label specifying one or more properties of the respective ISVD molecule.

34. One or more computer-readable storage media storing instructions that, when executed by one or more computers, cause the one or more computers to perform operations for predicting properties of an immunoglobulin single variable domains (ISVD) molecule, the operations comprising:
obtaining a token sequence representing the ISVD molecule;
generating an input vector by numerically encoding the token sequence representing the ISVD molecule;
generating an embedded feature vector by processing the input vector using an embedding machine-learning model having a first set of model parameters, wherein the first set of model parameters have been updated using self-supervised learning of a first machine-learning model that comprises the embedding machine-learning model and is configured to perform a sequence reconstruction task; and
processing the embedded feature vector using a property-prediction machine-learning model to generate an output that predicts one or more properties of the ISVD molecule, wherein the property-prediction machine-learning model has a second set of model parameters that have been updated using supervised learning, based on a plurality of training examples, of a second machine-learning model comprising the property-prediction machine-learning model, each respective training example comprising (i) a respective training input specifying a representation of a respective ISVD molecule and (ii) a respective label specifying one or more properties of the respective ISVD molecule.
